(19) United States
(12) Patent Application Publication
(10) Pub. No.: US 2017/0135654 A1
(43) Pub. Date: May 18, 2017

(54) AUTOMATIC OR ASSISTED REGION OF INTEREST POSITIONING IN X-RAY DIAGNOSTICS AND INTERVENTIONS

(71) Applicant: KONINKLIJKE PHILIPS N.V., EINDHOVEN (NL)

(72) Inventors: FRANCOIS CORNELIUS JOHANNES VAN DAAL, EINDHOVEN (NL); MARKUS JOHANNES HARMEN DEN HARTOG, EINDHOVEN (NL)

(73) Assignee: KONINKLIJKE PHILIPS N.V., EINDHOVEN (NL)

(21) Appl. No.: 15/318,838

(22) PCT Filed: Jun. 23, 2015
(86) PCT No.: PCT/EP2015/064015

§ 371 Date: Dec. 14, 2016

(30) Foreign Application Priority Data
Jun. 25, 2014 (EP) 14173825.2

(51) Int. Cl. A61B 6/12; A61B 6/00

(52) U.S. Cl. CPC A61B 6/12; A61B 6/461; A61B 6/469; A61B 6/4441; A61B 6/481; A61B 6/487; A61B 6/504; A61B 6/503; A61B 6/5229; A61B 6/545

(57) ABSTRACT

An imaging system (IMS) that includes a functionality (100) to help a user adjust an imaging geometry relative to a region of interest (ROI). A graphics display (505, 405, 510) is generated and displayed on a screen (MT) to indicate in a current field of view (FOV) a current position of the ROI within said FOV and a protocol required position of said ROI.

### FIELD OF THE INVENTION

The invention relates to a method of operating an imaging system to visualize a region of interest, an imaging system for imaging a region of interest, to a computer program element, and to a computer readable medium.

### BACKGROUND OF THE INVENTION

Across a range of fields, for instance in the medical fields, x-ray or other imaging equipment is used. Imagery of the internal structure of objects such as a patient can be acquired for benefit of diagnostic purposes for instance. However useful the imagery it comes at the expense of exposing the patient to x-ray radiation which pose health risks on their own.

In a medical x-ray imaging system, a user (for instance a radiological technician or radiologist) can select essentially any angle, detector or examination table position to visualize a specific region of interest under X-ray. In certain procedures, imaging protocols are prescribed for visualizing a specific anatomy. That is, angulation and rotation of the system are set in accordance with preferred protocols, ensuring that the anatomy of interest (e.g. in coronary angiography, specific coronary vessels or the current position of a catheter) is displayed in an optimal manner.

Unfortunately it has been found that in spite of a multitude of adjustment possibilities afforded by modern imaging equipment it still happens that the resulting images do not capture completely the region of interest. Consequently, re-adjustment of the imaging geometry is frequently required. This re-adjustment has been found to compromise image quality and also has been found to expose the patient to unnecessary x-ray dosage.

### SUMMARY OF THE INVENTION

There may be a need in the imaging art to aid a user operating an imaging system. The object of the present invention is solved by the subject matter of the independent claims where further embodiments are incorporated in the dependent claims. It should be noted that the following described aspect of the invention equally apply to the imaging system, to the computer program element and to the computer readable medium.

According to a first aspect of the invention there is provided a method of operating an imaging system to visualize a region of interest, ROI, of an object, the method comprising the steps of:

receiving i) prior information on a current position of the ROI within a current FOV and ii) information on a predefined position for said ROI within a predefined FOV;

establishing whether, in the current FOV, the two positions match;

if no match is established, displaying a graphical indicator to indicate to a user an instruction how to change a current imaging geometry of the imaging system so as to then achieve a repositioning of the ROI at the predefined position within said current FOV or within a new FOV.

Preferably, if a match is established, a graphical indicator may be shown indicate to the user that the two manners of representation match.

The imaging geometry (expressible in terms of one or more imaging coordinates) defines a spatial configuration of the imaging system relative to the ROI.

The “predefined position” indicates where the ROI is represented within a predefined (ideal) FOV whereas the “current position” is the position at which the ROI is displayed within the current FOV. The predefined position may be part of a protocol associated with the ROI one wishes to image. In other words, the protocol may prescribe in which part of the FOV the ROI, for example, a footprint of an object such as a catheter, is to appear when displayed, that is, the relative position of the ROI within the FOV is part of the protocol.

In addition to the ROI position, the protocol may prescribe the magnification level of the ROI, or any other visualization characteristic.

In case of incorrect manner of (ROI) representation, the displayed instruction informs the user on how to adjust the current imaging geometry so as to achieve the correct manner of representation. For instance, the user may be informed (by an “arrow” or other suggestive graphical symbology) to change the imaging geometry by panning or shifting the ROI or object by moving a support device (e.g., examination table) on which the object/ROI resides to achieve the required manner of representation in the current FOV. Alternatively or in addition thereto, the user may effect the change in imaging geometry by moving a gantry of the imaging system relative to the ROI/examination table to change the current FOV into the new FOV in which the ROI will then be displayable in the correct manner.

In other words the method helps the user to adjust the imaging system to predefined requirements/protocol. The adjustments can be done outside the actual X-ray image exposure period. This helps increase image quality. Also the system helps lower patient dosage because, during an interventional phase, imaging re-runs due to incorrect imaging geometry can be avoided. In particular, the method helps address the frustrating situation where operators (“user”) of imaging system find during an interventional procedure involving X-ray exposure that the FOV does not capture the ROI in its entirety.

Applicants have found that this unexpected mis-alignment comes about because the ROI is sometimes not precisely positioned from the outset at the iso-center of the imager. Another complication is that the patent-specific ROI under consideration may vary more than expected from the statistical assumptions that underlie the imaging protocol. Also, it has been found that in addition to the angulation and rotation, the position of the imager's gantry (e.g. c-arm) with respect to the table also influences the field of view.

Thus, if the system is not in an optimal starting position, the anatomy of interest may not be imaged completely. Re-alignment of the system may be required during an intervention, e.g. using fluoroscopy, causing an additional x-ray dose to the patient that should be avoided.

The proposed method allows an intuitive and quick way to adjust the imaging geometry for these variations and inaccuracies prior to a (high dosage) X-ray exposure, in particular prior to an intervention involving X-ray exposure.

According to one embodiment, a change of the imaging geometry is effected according to the indicated instruction. This change is responsive to a control signal issued either from the user or is issued automatically by the imaging system. Also, the change itself occurs either automatically without user interaction, or the user actively effects the change by moving the examination table for instance where the object resides.

According to one embodiment, a warning signal is issued if no match is established. In other words, a visual, auditory or other warning or alert signal is issued if the current manner of representation deviates from the predefined manner of representation or if the current manner of representation deviates by more than a predefined error margin.

According to one embodiment, the change of the imaging geometry is effected by any one or a combination of i) moving the object or ROI (e.g. by moving the examination table), ii) moving a detector or a probe and/or an X-ray source of the imaging system, iii) a collimation operation or iv) adjusting a radiation sensitive surface of the detector.

According to one embodiment, the imaging system is an interventional X-ray system, in particular a C-arm type X-ray imager. Preferably, the imaging geometry changeover occurs whilst there is no X-ray exposure.

According to one embodiment, the current FOV is based on a previously acquired image. The current FOV may be based on one or more a previous fluoroscopic frames, for instance the most recent LIH (last-image-hold) frame, or may be based on a previously acquired higher dosage (as compared to fluoroscopic dose) X-ray image.

According to one embodiment, in addition to matching the current and predefined position of the ROI within the FOV, the data processor is configured to match a current and a predefined magnification level of the ROI, that is, the desired magnification at which the ROI is to be shown. Adjustment of the latter can be achieved for instance by changing the SID and/or the detector format. The detector format defines the size of the radiation sensitive area of the detector with, for instance, “smaller area” amounting to larger magnification, etc.

The current position of the object (patient) and hence the ROI is based on analyses of previous images, current (geometry) system information and/or other a-priory knowledge.

According to one embodiment, a grid structure of segments or quadrants may be associated with the FOV, and the predefined position is associated with a specific one of these quadrants. Preferably, the data processor is configured to determine in which quadrant the current position is located, and establish whether this quadrant matches with the quadrant of the predefined position.

Alternatively any other suitable divisional component may be used for full or partial tessellation of the FOV.

The graphical indicator how to change the imaging geometry may include circle or disks or other position tracker symbology arranged, which may be superimposed on the current FOV or as a separate indicator. The indicator may be shown in addition to a visualization of the grid of segments, or combined therewith. Further, there may be directional components such as an arrow pointing from the current position ROI position to the required ROI position.

In short what is proposed herein according to one embodiment is a tool to aid the user in setting a correct starting position of the imaging system in respect of an object (or part thereof) to be imaged and to quickly find the correct FOV for a given ROI. The field of view of the system may be suitably divided into segments (quadrants). For each imaging protocol, one of the quadrants may be designated as a preferred start position. Then, before starting the X-ray imaging, fluoroscopy is used to record movement of a catheter tip. The system then determines in which quadrant the catheter tip is located and compares this to the preferred quadrant according to the protocol. If there is a misalignment, a visualization is provided to the user in the (recorded) fluoro image. While x-rays are off, the catheter tip may then be panned (manually or automatically) into the correct quadrant. Thus, proper alignment for the following CA may be ensured.

### DEFINITIONS

“FOV”: the irradiated volume in space as defined by a set of imaging geometry coordinates or parameters or settings. In other words, anything in this volume has at least the potential to be encoded in an image when acquired in said FOV.

“ROI”: a part of the object/anatomy one wishes to image or the position of a tool (e.g. catheter) or its footprint in the FOV.

“(Imaging) protocol”: a set of imaging geometry parameters that define a preferred (“best”) FOV for an anatomical ROI and/or for a position of the tool. In other words, each ROI is associated with one or more sets of imaging geometry parameters. The preferred manner of ROI representation and/or predefined FOV is based on the operator's workflow/preference or is based on a model of the target anatomy and a specific clinical task to be performed at said ROI.

### BRIEF DESCRIPTION OF THE DRAWINGS

Exemplary embodiments of the invention will now be described with reference to the following drawings wherein:

FIG. 1 shows a block diagram of an x-ray imaging system;

FIG. 2 shows diagrammatically different imaging protocols;

FIG. 3 shows a display of a field of view;

FIG. 4 shows a series of field of views;

FIG. 5 shows a graphical indicator superimposed on a field of view;

FIG. 6 shows a sequence of field of views each superimposed with a graphical indicator;

FIG. 7 shows a flow chart of a method of operating an imaging system.

### DETAILED DESCRIPTION OF EMBODIMENTS

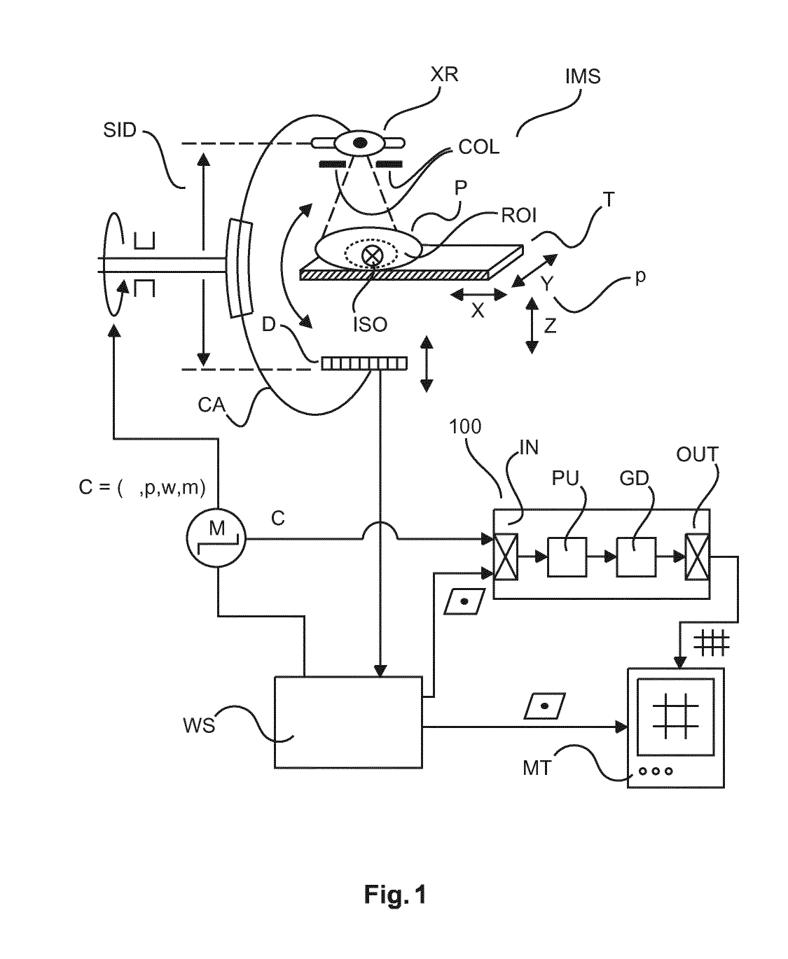

With reference to FIG. 1, there is shown a schematic block diagram of an x-ray imaging system IMS. Basic components of the imaging system IMS (also referred to as “imager” herein) includes a gantry such as a rigid C-arm CA that carries at one of its ends an x-ray tube or source XR and on the other end a detector D or image intensifier.

The x-ray source XR is configured to emit, during an imaging session, x-ray radiation. More particularly, the X-ray beam passes through an examination region and then impinges on a radiation sensitive surface of the detector D. Within the examination region traversed by x-ray beam XB is an examination table T. On the examination table T, a sample/object P to be imaged (for instance, a human or animal patient) is deposited.

When correctly positioned, the x-ray beam passes through patient P at a region of interest ROI, e.g. the human heart or other organ or part thereof. During its passage through the sample P, the x-ray beam is modified for instance by absorption interaction within matter in the sample P. The degree of absorption is a direct measure of the absorption co-efficient distribution or of the density distribution in the patient. Individual rays of the x-ray beam are therefore differently modified or absorbed depending on where the respective rays pass through the sample P. The so modified x-ray beam that emerges at the other end of the patient then interacts with the detector. Depending on the intensity detected at the detector, corresponding electrical signals are issued which are then passed to a DAS (data acquisition system—not shown). The DAS includes suitable A/D conversion circuitry to convert said electrical signals into digital form, also referred to as detector raw data, essentially an array of numbers. A work station WS is communicatively coupled with the DAS. The raw detector data is transmitted to work station WS via a wired or wireless connection. There, the raw detector data may then be stored away for later reference or is processed by suitable visualization software into images which can be rendered for view and displayed on a monitor MT. The raw data may also be directly applied to an image intensifier screen to so furnish real-time imagery, for instance during a fluoroscopy.

The x-ray images produced by the imager IMA are essentially projection views on the imaged sample acquired at a certain projection direction relative to the region of interest ROI. The projection direction (which may be taken as the direction of a central beam of the x-ray beam relative to the region of interest) is adjustable so that one or more projection images along different projection directions can be acquired of the ROI. Also, by adjusting for the projection direction, the best “view” on the ROI for a given clinical task can be chosen.

The different projection directions are assumed by changing the spatial configuration of the imaging system IMS. Different spatial configurations for the purpose of image acquisition are referred to as “imaging geometry”. One way to change the imaging geometry is to have the C-arm CA rotatably mounted, as is the case in a preferred embodiment, although this is not limiting. In one embodiment, rotation is around two or more rotational axes. According to one embodiment there are two rotational axes shown by the two curved arrows in FIG. 1. In this way the detector and/or the x-ray source can orbit on an imaginary sphere around a center at which, ideally, the ROI is situated. The center of this imaginary sphere is also referred to as the iso-center ISO of the imaging system IMS. As shown in FIG. 1, one of the rotation axes extends into the paper plane (shown as a little cross at ISO) whereas the other axis runs parallel to paper plane. As a matter of convention, rotatory displacement around one of the axis is referred to as “rotation” whereas rotatory displacement around the other axis is referred to as “angulation”, both specifiable by two angular values.

In one embodiment, apart from or instead of the two rotational degrees of freedom, there are other mechanical degrees of freedom envisaged to adjust the imaging geometry relative to the ROI to be imaged. Preferably, the number of mechanical degrees of freedom of the imager IMS is such that a projection image at essentially any desired projection direction is acquirable. This flexibility allows examination of the ROI from the (e.g., clinically) most relevant direction.

Other options for changing the imaging geometry includes in one embodiment adjustability of the SID, that is, of the distance between the x-ray source XR and the detector D. There may also be a panning functionality so that, for instance, the examination table T (and hence the ROI) can be panned in a plane past a given C-arm position along either one or both of panning directions X and Y. Other panning functionalities may include translation of the C-arm itself relative to the table/ROI. In one embodiment a given rotation and angulation can be “locked” to maintain same during the translation. Also the height of table T along Z direction is adjustable in one embodiment. Other options to change the imaging geometry include collimator C adjustments. In one embodiment, collimator C is essentially a set of blades which are moved into or out of the beam to more or less restrict the x-ray beam to a desired width. In one embodiment, the radiation surface of the detector can also be changed by disabling or enabling select detector pixels to so define or restrict the maximum possible field of view, given by the number of rows and columns of detector pixels. Any combination or any sub-combination of the previously mentioned imaging geometry adjustment options are envisaged herein. In other words, it will be appreciated that the number of degrees freedom enjoyed by the IMS as per FIG. 1 is purely exemplary. Other systems are envisaged that afford fewer degrees of freedom or a higher degree of freedom than shown in FIG. 1.

A given imaging geometry relative to the iso-center ISO of the imaging system can be conveniently described in numerical form by imaging (geometry) co-ordinates/parameters C. Using the system of FIG. 1 as an example, imaging co-ordinates C may include an angular part α=(α₁,α₂) (that describe rotation and angulation), a panning part p=(x,y) that specifies the panning or table position T relative to the x-ray beam. There may also be a window size part w that describes the size of the volume in cross section that is irradiated by the x-ray beam (this may include a set of collimator blade/leaf positions or an array of flags that defines which detector pixels are to be enabled, etc.). Finally, there may also be a magnification part m formulated for instance in terms of z to define the SID, for instance. The collection of any given set of imaging coordinates C define a field of view (FOV) of the imager IMS. It defines the spatial region (usually a cone) in the examination region that is “floodable” with (primary) radiation in an exposure when the X-ray source is energized to emit radiation.

Any given set of imaging geometry co-ordinates C corresponds to certain control signals that can be issued by suitable control module and associated control circuitry and/or interfaces. The control module may reside on the work station, for instance. The control module allows to at least-semi automatically changing the imaging geometry. The control module is communicatively coupled (in a wired or a wireless fashion) with one or more actuators M of the imaging system IMS. When instructed by the control module, actuators M operates (mechanically, electro-mechanically or electronically) to change the imaging geometry into the desired one. Adjustment of the imaging geometry is either purely manually (e.g., by a handwheel or lever actuator mechanisms, etc.), or is semi-automatic. In the earlier case, it is envisaged in one embodiment that suitable electronic tracking modules are in place that by sensor action or otherwise track the handwheel motion to track the imaging geometry coordinates. In the semi-automatic embodiment the user of the imaging system uses suitable user interface tools such as a joy stick arrangement to ask for specific imaging coordinates. Corresponding control signals are then issued in response to joystick use to effect the desired change of the imaging geometry. There is also a fully automatic embodiment, where an imaging protocol (essentially program instructions) is are read-in, translated into a suitable sequence of control signals which are then issued to the actuators M to adjust the imaging geometry of the IMS into the desired one. Whether in the manual, semi-automatic or automatic embodiment, it is assumed herein that the imaging system IMS is “aware” (by operation of a suitable tracking/sensor functionality) at any time of its current imaging geometry (and possible of the previous imaging geometries) and it is assumed herein that the current or previous imaging geometries can be queried if desired. This can be achieved, for instance, by logging the respective imaging coordinates during the various imaging geometry changes.

The x-ray imaging system IMS as briefly described above can be used in a range of different applications in the medical or non-medical field (such as for instance non-destructive material testing or baggage scanning).

In the following, a medical use scenario (cardiac interventions) will be explained for exemplary purpose that is not to limit the invention. In cardiac interventions, a catheter is introduced into the patient. The catheter is then advanced through the human vasculature until a lesioned site in the cardiac vasculature is reached. Once the catheter tip has arrived at the desired location in the human heart, treatment at the lesioned site (for instance, a stricture) can be treated by a suitable medical tool, such as a balloon catheter etc.

Unfortunately, the vasculature tissue itself is not visible in x-ray images unless steps are taken. The radio-opacity of the vascular tissue as such does not form sufficient contrast. In order to still achieve sufficient contrast, a contrast agent is administered to the patient. Administration of the contrast agent is via the catheter once it has arrived at a desired starting position in the vasculature. If the catheter is at the desired starting position, a predefined quantity of contrast agent is discharged at the catheter tip and then profuses into relevant parts of the vasculature. At this time, x-ray images (angiograms) are taken which will then present a projection view on the cardiac vasculature at the desired contrast. In this manner, the extent of the lesion (e.g., stricture) or other anatomical relevant information can be obtained by a medical professional from this angiographic imagery.

The intervention procedure, as briefly sketched above, includes, in terms of imaging requirements, two main phases, a navigation phase and an operational or interventional phase.

During the navigation phase the catheter is navigated, that is, urged by medical staff through the vasculature towards the desired starting position. Once at the desired starting position, the interventional phase commences, for instance the contrast agent is administered, the site is x-ray imaged, or treatment tools (balloon catheter, etc.) are deployed. Different types of images are acquired during those two phases. During the navigation phase a fluoroscopy is carried out. Under fluoroscopy the x-ray tube operates at a relatively low x-ray dose so the images have a relatively low contrast. Images are acquired at a suitable frame rate, at, for instance, 15 frames per second. In this manner, a “cine” view on screen MT is formed that allows the user to visually follow in real time the progression of the catheter through the patient. In this manner, the user is informed about the location of the catheter tip inside the body. Due to the relatively high radio-opacity of tip, there is still sufficient contrast so the user is safely informed about the position of the catheter in the body.

The imagery in the fluoroscopic phase is distinguished from imagery acquired in the interventional phase. In the interventional phase, the images are acquired with a higher x-ray dose than in the fluoroscopic phase to so afford better contrast. This is because images acquired in the interventional phase are for diagnostic purposes where it is not merely the position of the catheter tip that is of interest. In the interventional phase one wishes to examine for anatomical details that would not be discernable from low dosage fluoros. In other words, the diagnostic X-ray images are, in terms of dose, more costly than the lower dosage fluoros but afford more structural detail. Owing to this imaging workflow distinction, we will use in the following the term “(diagnostic) x-ray image” for a high dosage image (acquired in the interventional phase), whereas the term “fluoro” is used for an image acquired (in the navigation phase) at a lower dose than that used for x-ray images in the operational phase.

The mentioned (“right”) starting position for the catheter may be prescribed by an imaging protocol for a given anatomical part (region of interest—ROI). Once this starting position is assumed, diagnostic image acquisitions in the operational phase of the intervention can commence. In other words, the protocol encodes knowledge about the correct imaging co-ordinates C (and hence FOV) to be used for a specific anatomy part. This knowledge may be derived from investigations of a large number of previous patients to deduce certain assumptions of an average anatomy. For instance, in one embodiment, cardiac sample measurement can be consolidated into an average geometrical model. Geometrical rays can then be cast thereacross to render different projection views that can be examined to find the best possible imaging coordinates or FOV for any given ROI. The respective ROI can be tagged by a code or otherwise identified and said code may then be stored (in a database) in association with the best imaging coordinates C_{ROI} so found. This association forms a basic imaging protocol. The “best” imaging coordinates can be queried using the code to retrieve, from a medical knowledge database, the best FOV/imaging coordinates for a given imaging session.

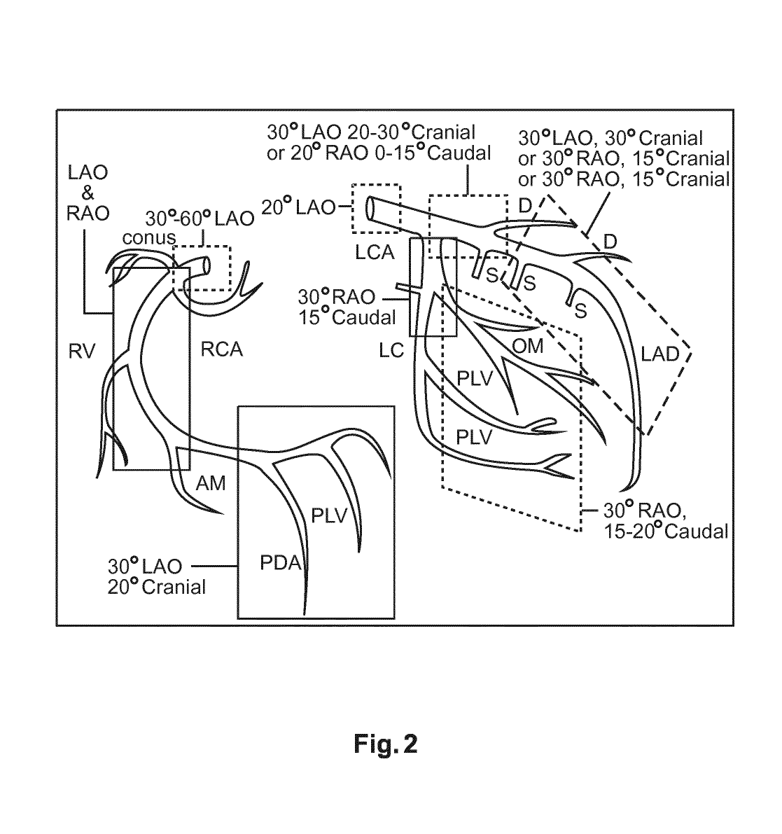

FIG. 2 depicts diagrammatically different coordinate based protocols. The left hand side of the diagram shows branches of the right coronary artery RCA whereas the right hand side of the image shows the left coronary artery LCA. For instance, if one wishes to image the branches OM (obtuse marginal) or PLV (posterior left ventricular) of the LCA, the protocol prescribes imaging co-ordinate such as Right Anterior Oblique ROA at 30° and Caudal at 25° as the preferred FOV and hence start position for the catheter position to best image the OM or PLV branches. The predefined FOV according to the protocol may also be referred herein as the “protocol FOV”.

When using the imaging coordinates as per the protocol to adjust the imager IMS's geometry, it is assumed that the region of interest ROI is accurately positioned in the iso-center ISO of the imager. However, as a matter of practical reality, this may not always be the case and the ROI may be slightly off the iso-center ISO. As a consequence, the field of view will be slightly off, and possibly crucial anatomic information may be missed out on. Information may also be outside the FOV if the specific patient to be imaged has an anatomy that is not within statistical variance of the average anatomy model on which the protocol is based.

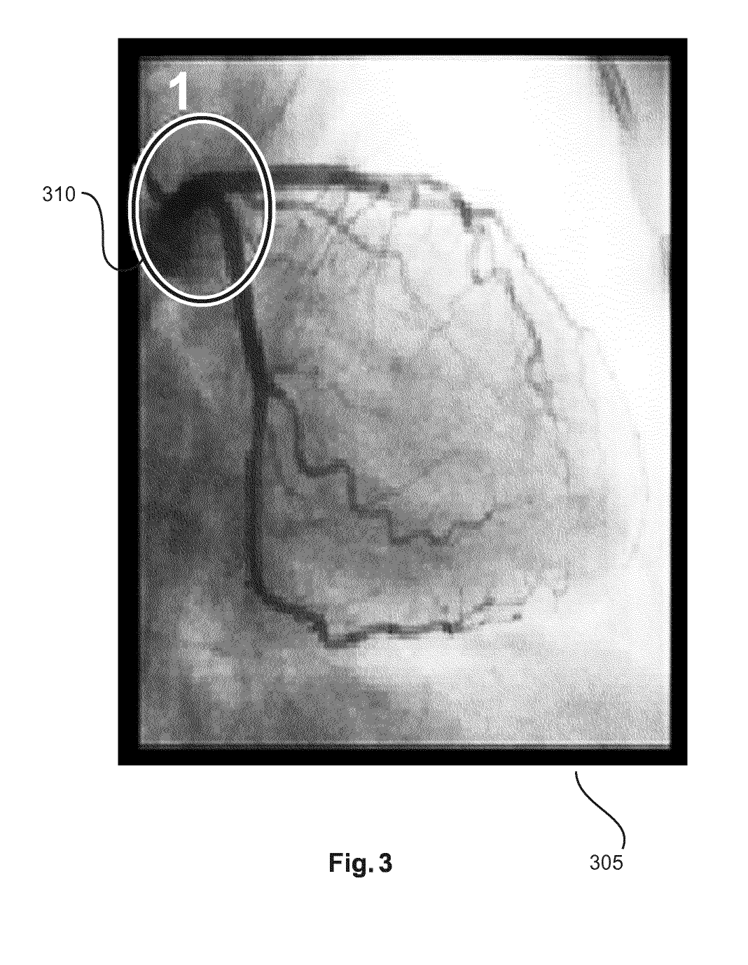

FIG. 3 shows a protocol FOV 305 that has been acquired for the OM and PVL branches of the LCA using the co-ordinates mentioned above. If the patient has been positioned correctly relative to the iso-center and their anatomy is an average one, then one would expect the correct position 310 for the catheter tip to be at the upper left hand corner of said protocol FOV 305. In other words, one can think of a grid system made up of suitably numbered quadrants structuring the protocol FOV. For instance, for the protocol FOV shown in FIG. 3, one can say that the correct start position 310 is in the first quadrant shown as a circle 310 in FIG. 3 of the FOV 305. In yet other words, the protocol FOV prescribes where (that is, in which image portion e.g., “quadrant”) in the FOV the catheter tip's footprint should be situated.

Additional representational requirements may for instance be the “size” of the catheter tip and/or the vessels which requires acquisition at a certain SID. These representational requirements may differ from application to. These representational requirements may be likewise encoded in the data structure of the protocol, for instance as a code for the number of the quadrant for the expected position. It will be understood that using the catheter's tip is merely one exemplary manner to define the catheter position and with other tools other salient parts may be used instead to define a position of said tool.

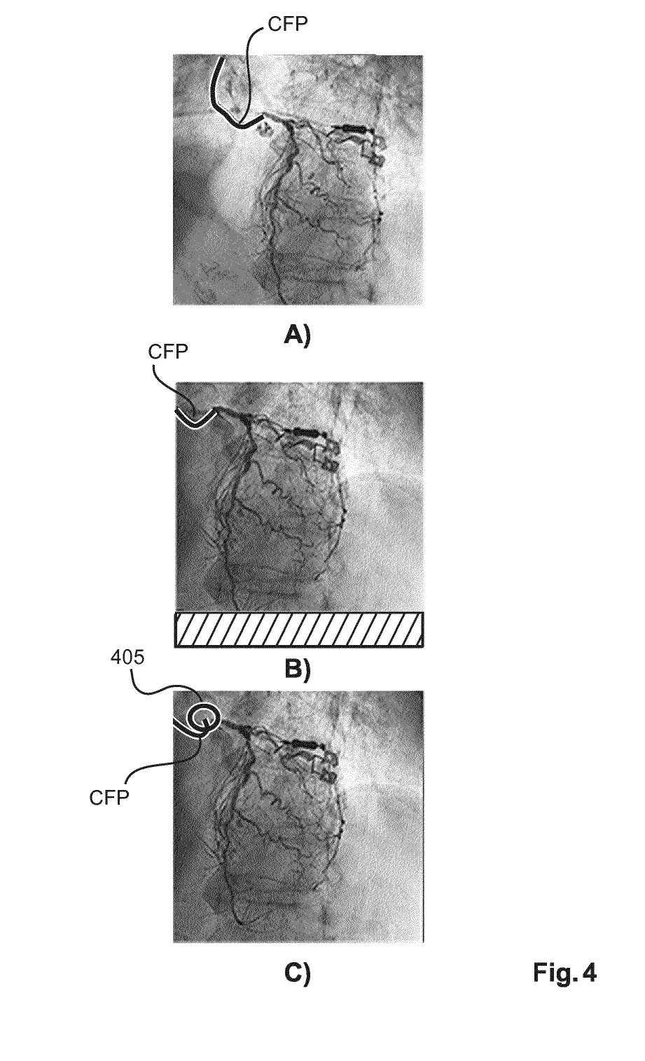

FIG. 4 is an illustration of what can go wrong during a diagnostic imaging run in the interventional phase. The FOV as per FIG. 4A at the very left shows the FOV at the start of an angiographic exposure run image to capture the left coronary artery during contrast agent perfusion. In other words the frame in FIG. 4A shows the first acquisition during the diagnostic run.

FIG. 4B shows the end of the angiographic exposure run. As can be seen in frame 4B, there is a hatched region at the bottom of the frame that represents the anatomy that has not been captured due to an incorrect start position initially at frame A). In other words, not the whole of the left coronary artery can be captured because some of the anatomy of interest was outside the currently irradiated FOV although, on the face of it, the correct imaging co-ordinates as prescribed in the protocol have been used. Now, the user, in an attempt to still capture some of the out-of FOV vasculature may shift the examination table or may otherwise adjust the imaging geometry during the acquisition in the diagnostic run thereby compromising image quality as shown in frame 4C at the right hand side of FIG. 4.

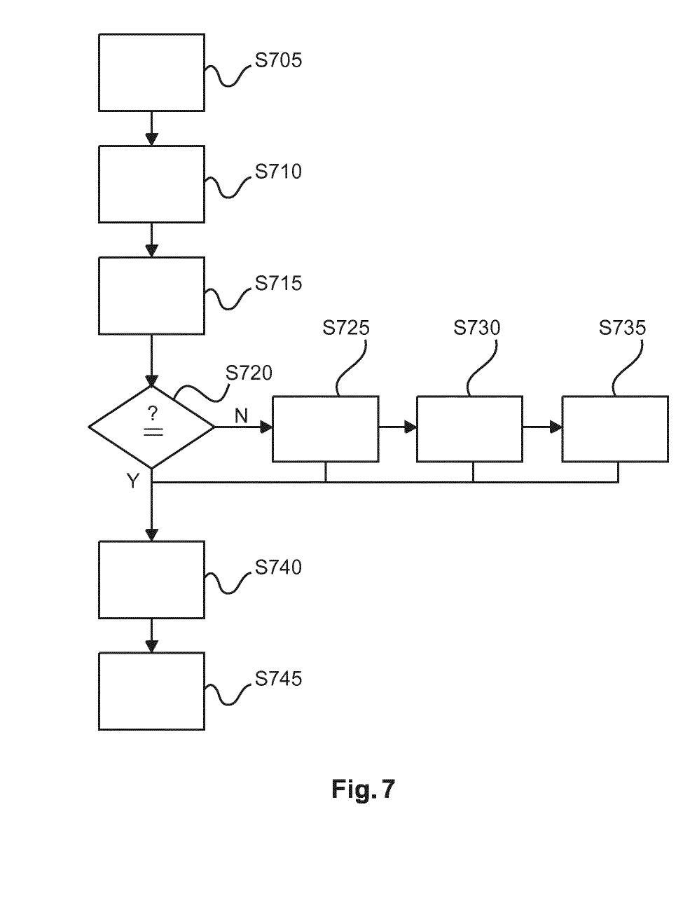

To avoid the situation as per FIG. 4 C) or similar, it is proposed herein an imaging system that includes a help functionality as per an image and/or coordinate-data processing apparatus 100. The image processing apparatus may serve as a tool (and a related method—see FIG. 7 below) to aid the user in finding the correct starting position using preferably only information available prior to the (next) diagnostic high dosage exposure. For the prior information may comprise the image geometry coordinates generated by the imager IMS and/or the available fluoroscopy frames from the latest of previous navigation phase to compute the correct start position and to indicate it graphically to the physician or at least alert the user if the correct start position is not assumed. The proposed apparatus 100 may be configured to operate as an add-on with existing imaging systems. Panning or other imaging geometry changes during the high-dosage exposures in the operational phase can be avoided which in turn helps maintain image quality without incurring extra patient dosage.

Referring back to FIG. 1, the proposed image processing apparatus 100 comprises input IN and output OUT interfaces, a data processing system PU and a graphics generator GD according to one embodiment. Very broadly, the apparatus receives a definition of the protocol FOV (as selected by user request) Also, not necessarily at the same time, the apparatus receives imaging geometry co-ordinates as generated by the imaging system and/or (preferably the latest available) image information, such as a previous (for instance latest) fluoroscopic frame information. The processing unit PU then computes the expected, according to protocol, correct starting position in the field of view, i.e. the predefined position of the ROI including the catheter tip within the field of view, and computes the latest known, or a previously available, position of the catheter tip, i.e. the current position of the ROI including the catheter tip within the field of view

Either one or both positions can be encoded by one or more suitable graphical indicators which are used to form a graphics display. The graphics display is then forwarded by graphics display generator GD via output interface OUT and is then superimposed to a current field of view. In addition to the graphical display generator, the system may also include a transducer to produce an alert signal (visual or acoustical) to alert the user when the current or the latest available position of the catheter tip in the current FOV is not according to protocol.

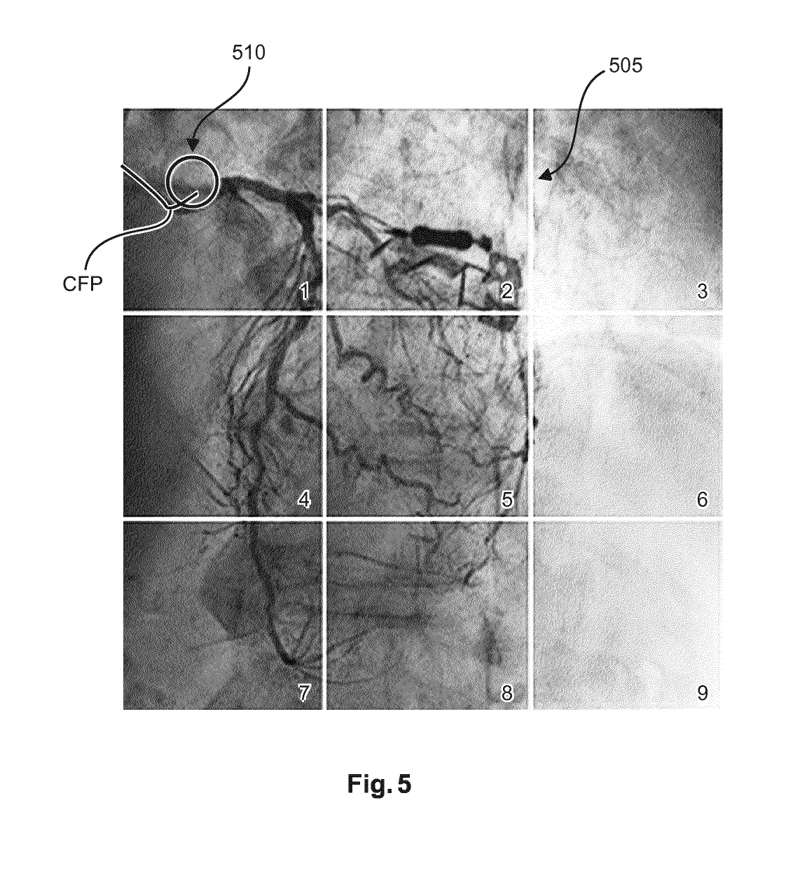

With reference to FIG. 5 there is shown a graphics display generated according to one embodiment. The graphics display is shown as superimposed on an existing/current X-ray image covering a field of view. A number of segments, for instance quadrants numbered 1-9, are associated with the field of view, said quadrants structuring the field of view into different spatial portions. The segments formed by quadrants form a 3×3 grid 505 for example. The graphics display includes a visualization of the quadrants 1-9.

In one embodiment, there is also a separate graphical indicator 510 in addition to grid 505 for indicating the predefined start position according to protocol. The graphical indicator 510 for the ideal starting position is suitably color- and/or shape-coded. The FIG. 5 embodiment shows the graphical indicator 510 as a circle. Other shapes such as triangles, squares or otherwise are also envisaged herein. In the example shown in FIG. 5, the correct quadrant for the expected start position of the catheter tip is in the first quadrant in the upper left hand side corner. The situation as depicted in exemplary FIG. 5 is one where the footprint CFP of the catheter tip happens to be in the correct quadrant 1. The graphics display widget is displayed prior to the diagnostic image run and is superimposed for instance on a fluoro frame, preferably the latest available.

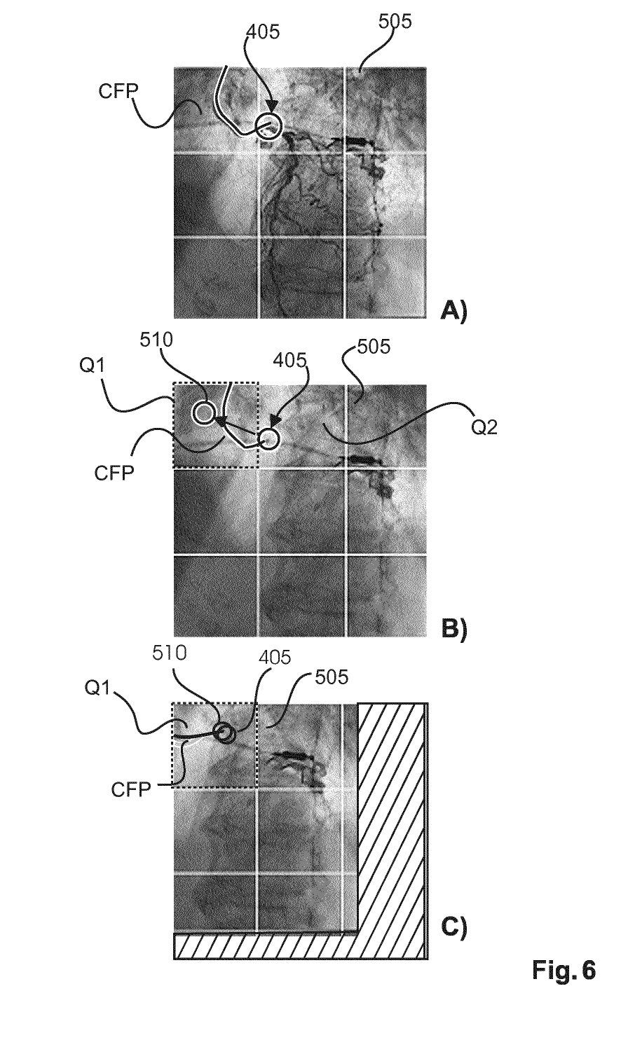

Reference is now made to the three panes A)-C) in FIG. 6 that illustrate a use scenario of the graphics display 505. In one embodiment, during the fluoroscopic low dosage image run, the catheter's tip is tracked across the frames by suitable segmentation software. This can be done because the appearance of the catheter in the projection images are a-priori known as the structure of the catheter is known. The respective positions of the catheter footprint can be indicated by a tracker indicator 405 shown exemplary as a circle in pane A) as reference numeral. The FOV as per A) also includes a 3×3 grid 505 generated by the graphic display generator GD and superimposed on each of the sequential frames.

Pane B) of FIG. 6 shows the last image in the fluoroscopic run with grid 505 and the latest catheter tip as shown by tracker symbol 405 superimposed on same. This last or latest fluoro frame can be obtained by a last-image-hold (LIH) functionality, which operates to essentially “freeze” the display at the latest frame of the fluoroscopic run. In the embodiment of FIG. 6 B), the grid further includes a graphical indicator symbol 510 as a circle in quadrant Q1 to indicate the expected or predefined “best” start position 405 for the tip CFP according to protocol.

In FIG. 6 B), the predefined position is additionally indicated by graphically encoding the relevant quadrant Q1, e.g., by color or line type. For instance, in the FIG. 6B) embodiment, quadrant Q1 is shown in dashed lines, to indicate that it includes the predefined or required tip position. In one embodiment, the quadrant Q2 holding the current tip position may be differently graphically encoded. In this manner, the graphics display may be formed solely as a gird with no additional tracker indictor symbols 405,410. In this grid-only embodiment, the two tip positions (that is, the required one and the current one) are indicated by different graphical renderings of the two respective quadrants that happen to include said positions.

The FOV in pane B) represents a scenario where there is a deviation between where the tip currently is and where it should be according to protocol. The processing unit operates to compute instructions on how the current FOV needs to be changed so that the tip CFP is displayed in the correct quadrant, in this case Q1. The instructions correspond to a change of the imaging geometry, for example the imaging coordinates. The instructions can be rendered graphical to bring the required change of the imager's geometry to the user's attention, for instance by superimposing the grid and or tracker indicators. Alternatively or additionally, an arrow or other directional symbology may be displayed to indicate the required panning operation or corrective action. Additionally, a dedicated flash lamp can be activated and/or a warning sound may be issued by a speaker system to alert the user that a corrective imaging geometry change is called for.

In other words, the graphics display informs the user that the panning motion is required so as to move the catheter tip from its current position within the second quadrant Q2 of the field of view over into a predefined position within the first quadrant Q1 of the field of view.

The motion of the catheter tip can then be effected by moving the catheter tip itself, e.g. by panning the examination table or by translating the C-arm. In either case imaging information as per the current FOV will be lost during the changeover to the new protocol FOV. The loss of image information is shown as the hatched area in frame C). After the panning, the catheter tip position is now situated in the correct first quadrant as shown in pane C) of FIG. 6. This positional correction of the region of interest has been carried out entirely based on already available information, that is, information that was available prior to the commencement of the next diagnostic imaging run. Also the panning or corrective action was carried out whilst there was no high dosage diagnostic exposure. After the change in imaging geometry and once the catheter appears (in the current or updated FOV) at the correct start position, the diagnostic high dosage x-ray exposure can now commence. It is now ensured that the entirety of the anatomy of interest can be captured.

Reference is now made to the flow chart of FIG. 7 detailing the various steps for adjusting a position of a region of interest in a field of view.

In step S705 a desired image protocol for a certain part of the sample under consideration (for example, a certain anatomy of the cardiac-vasculature) is selected. The protocol calls for a predefined protocol FOV defined by a set of imaging coordinates. The imaging protocol may also prescribe a manner of representation for certain objects in said FOV, for instance in which portion of the FOV a ROI is to be featured. The ROI may include a position of a medical tool such as a catheter.

In step S710, information about a current manner of representation of the ROI in a current FOV is gathered. In one embodiment this is done by starting a fluoroscopic imaging sequence which then terminates in a last or latest image frame conserved by a last-image-hold functionality.

In one embodiment, the position of the ROI in the current FOV is established.

For instance, in one embodiment, the ROI (e.g., a catheter's tip) is tracked across the fluoro sequence and into the last frame so that the position in the latest available FOV is known. In this preparatory information gathering step S710, the information is collected before commencing image acquisition at a higher dosage than that used of the fluoro image run. Collecting this information may also include intercepting and processing the imaging co-ordinates that are generated by adjusting the image geometry for the current FOV.

The positional information gathered at previous step S710 is then received at a processing unit in step S715. In other words, the information available at step S715 is comprised of i) a current position of the ROI within the FOV and ii) a predefined or required position of the ROI (e.g. the catheter tip) within the predefined FOV. At step S720 the current and predefined positions within the FOV are compared. If there is a mismatch, instructions to change the current imaging geometry are computed, for instance in terms of imaging geometry coordinates. For instance, the table should by corrected for by offset (x,y) or the C-arm CA should be moved by (−x,−y), or the collimator should be opened up by certain amount, etc. The in-image coordinates of the ROI are compared with the predefined position as prescribed by the selected protocol. If the ROI essentially coincides with the predefined position or at least falls within a pre-set error margin thereof, this match is preferably indicated by setting a suitable flag for instance.

A grid or other partition or tessellation symbology could be used to define the various portions of the FOV. For instance, the predefined manner of representation associated with the selected protocol may require the ROI to fall within the (n,m)th-grid segment, e.g. quadrant. The in-image (in the current FOV) coordinates of a reference part of the ROI (e.g., the catheter's tip) is then checked whether they fall within said (n,m)-grid segment. If the ROI (as per its coordinates of the reference part) is found outside the predefined position and or said margin, this fact is indicted as a mismatch.

A similar comparison is carried out when the manner of representation tested for is that of magnification in addition to position. The size of the pixel-area of the ROI can be computed (after segmentation) to establish whether the SID is as prescribed. Alternatively, the current SID setting is requested from the imager's control module and compared with the predefined SID as per protocol in order to establish whether the correct SID is used.

Depending on whether or not there is a mismatch established in checker step S720, a corresponding graphical indicator widget is displayed as per steps S725 or S740 on the current FOV to inform the user about i) the outcome of the checker operation S720 and ii) what can be done to bring about a match if there is none.

If at step S720 a mismatch is established, the process flow passes on to step S725 where the displaying of a graphical indicator is effected to graphically render said instruction in the current FOV. The graphical indication of said instruction can be achieved for instance by the graphically rendering the previously mentioned grid structure, where the respective segments or quadrants themselves are marked up accordingly. For instance, the quadrant that includes the correct start position is highlighted in one color whereas another graphical segment that includes the current position of the ROI is indicated in a second color, or is rendered in a different line type, etc. A more “dynamic ergonomy” may be used instead or on addition to a mere static symbolic encoding of the mismatch in ROI representations. For instance, the two relevant quadrants Q1, Q2 may (also) be shown as flashing at different frequencies or only one is flashing whereas the other is not, etc. Alternatively, the grid structure may not be formed by solid lines but lines may be dashed or otherwise interrupted. In another embodiment the quadrants are merely shown by their corner points by a suitable cross symbol for instance.

Alternatively or additionally, the area within segments Q1, Q2 are filled in different hues or color but soft enough so as to not obscure image details. In another embodiment, dots, disks circles, “cross-hair” or other (point) tracker symbols are shown to indicate the two positions. The cross-hair symbols can be displayed in addition or instead of the graphical grid. Preferably both, the two different cross hair symbols are displayed together with the grid floating on same so as to form a common graphics display. Two tracker symbols “float” across the grid and are positioned in different quadrants (such as in FIG. 6B)) will then immediately and intuitively convey to the user the fact that a mismatch has been established as to where in the FOV the ROI ought to be versus where it actually is. In addition to the tracker symbols or along with same there may be directional components such as an arrow pointing from the current ROI position to the required ROI position.

In step 730, in response to control signal a corrective re-adjustment of the imaging geometry occurs to effect that the ROI will be displayed in the prescribed quadrant. For instance, catheter's position relative to the detector is moved for instance by table panning or translation of the gantry or re-adjusting the collimator blades until the graphical indicator indicates that the current position now essentially coincides with the prescribed position. Depending on the manner or representation one wishes to correct for, one may also re-adjust the SID or take any other corrective action. It is understood that, during the re-adjustment of the imaging geometry, the graphical indicator is dynamically adapted. For instance, the tracker point symbol 405 for the current tip/ROI position is shown to move across the grid towards the tracker symbol or quadrant that represents the correct, that is, the predefined position as per protocol.

The change in imaging geometry can either be done automatically in which case the instructions are forwarding a to a suitable control module. The control module then instructs respective ones of the actuators to carry out the panning motion of the examination table.

Optionally, there is a visual or acoustical or tactile (vibration of joystick for instance) alert signal generated at step S735, to alert the user that a mismatch has been established at step S720.

It is understood that the apparatus 100 operates to essentially freeze the grid on the screen MT as the user changes the imaging geometry to achieve the ROI ends up in the prescribed quadrant Q1. Visually then, the viewer will be under the impression as if the FOV, during its change (e.g., during gantry CA or table motion) is slipping past the grid from underneath it.

If, however, at checker step S730 it is found that the two representations do match, this fact is likewise graphically rendered in step S740. In this case, according to one embodiment, the two tracker symbols in the grid are displayed in the same quadrant or (in the embodiment without tracker symbols) the same quadrant flashes alternately in two different colors, each color understood to represent respectively one of the two positions. Alternatively, in the embodiment without grid, the two tracker symbols are shown as essentially superimposed or at least as overlapping to graphically render that a match has been found.

After step S740, or after the corrective action at step S730, the ROI (e.g., the catheter position) is now correctly positioned and the imaging system's geometry is now correctly adjusted to capture the anatomy of interest. Consequently, at step S745 the high dosage diagnostic image run (or if still required a fluoro run) can then safely commence.

It will be appreciated herein, that the above method may be based on previous imagery (of instance a fluoro sequence) which is the preferred embodiment, but alternative embodiments are likewise envisaged where for instance the checker step S720 operates “blindly” solely on imaging coordinates and coordinate sequence that help pinpoint the position if the catheter tip for instance. This can be achieved by using a robot for instance, that introduces and advances the catheter through the patient. The control signals generated by the robot can be related to the imaging geometry by definition of a suitable common coordinate system to track the position of the catheter tip throughout its journey. At one instance however some fluoroscopic imagery may still be required. But in either case, the proposed system does not rely on X-ray images acquired at a much higher dosage than the dosage used for the previous fluoros and the robot based “coordinates-only” variant of the method may even further reduce the number of fluoro frames required. The method of course does not excluded using high dosage imagery previously acquired, but the method does not rely on this high dosage imagery but can operate on either no imagery at all or can do with low dosage imagery. In other words still, the method helps adjusting the imaging geometry solely based in information (images and/or coordinates) before one commences a X-ray exposure using a higher dosage that that for the previous fluoros.

Although the above has been described primarily with reference to absorption based x-ray images, it is also understood that other techniques such as phase contrast imaging is, US imaging or MRT imaging are likewise included herein. Also, the above has been described for exemplary purposes only with reference to an interventional C-arm x-ray apparatus. However, this is not to limit the invention as the described apparatus and method can also be used with benefit in other imaging systems such as CT apparatus, MRT and ultrasound or in any other interventional imaging setting.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above-described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An imaging system for imaging a region of interest, ROI, of an object, comprising:
an interface configured to receive i) information on a current position of the ROI within a current field of view, FOV, and ii) information on a predefined position for said ROI within a predefined FOV;
a data processor configured to establish whether the current position and the predefined position match;
a display configured to display, if the data processor established there is no match, a graphical indicator to indicate to a user an instruction how to change a current imaging geometry of the imaging system so as to then achieve a displaying of the ROI at the predefined position within said current FOV or in a new FOV.

2. The system of claim 1, wherein
the display unit is further configured to display, if the data processor established a match, a graphical indicator to indicate to the user that the two manners of representation match.

3. The system of claim 1, wherein the data processor is further configured to established whether a current magnification level in the current FOV matches with a predefined magnification level in the predefined FOV.

4. The system of claim 1, further including a support device on which the object resides, wherein the support device is configured to be moved in accordance with the instruction so as to effect the change of the imaging geometry.

5. The system of claim 1, wherein the system is an interventional X-ray imaging system, comprising a C-arm mounting an X-ray source and an X-ray detector.

6. The system of claim 5, wherein the C-arm is configured to be moved in accordance with the instruction so as to effect the change of the imaging geometry

7. The system of claim 1, wherein the interface is configured to receive an imaging protocol, the predefined position of the ROI being associated with said imaging protocol.

8. The system of claim 1, wherein the data processor is configured to associate a grid structure of quadrants with the FOV and to establish whether a quadrant in which the current position is located matches with a quadrant in which the predefined position is located.

9. The system of claim 1, wherein the predefined position corresponds to a starting position of a tip of a catheter to be used in an interventional procedure.

10. A method of operating an imaging system to visualize a region of interest, ROI, of an object, the method comprising the steps of:
receiving (S715) i) prior information on a current manner of representation of the ROI in a current FOV and ii) information on a predefined manner of representation for said ROI;
establishing (S720) whether, in the current FOV, the two manners of representation match;
if no match is established, displaying (S725) in the current FOV a graphical indicator superimposed on same to indicate to a user an instruction how to change a current imaging geometry of the imaging system so as to then achieve a displaying of the ROI in the predefined manner of representation in said current FOV or in a new FOV; or
if a match is established, displaying (S740) in the current FOV a graphical indicator superimposed on same to indicate to the user that the two manners of representation match.

11. A computer program element for controlling a system which, when being executed by a processing unit is adapted to perform the method steps of claim 10.

12. A computer readable medium having stored thereon the program element of claim 11.
